(19) United States
(12) Patent Application Publication
(10) Pub. No.: US 2004/0247542 A1
(43) Pub. Date: Dec. 9, 2004

(54) Cosmetics for ultraviolet light protection

(76) Inventor: Masaakira Horino, Kanagawa (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/811,231

(22) Filed: Mar. 29, 2004

(30) Foreign Application Priority Data
Jun. 9, 2003 (JP) 2003-163893

(51) Int. Cl. A61K 7/42

(52) U.S. Cl. 424/59

(57) ABSTRACT

A dispersion which has a stable dispersibility, that is, a dispersion in which an ultraviolet light scattering agent is dispersed homogeneously and with good stability for a prolonged time with a dispersant, and a cosmetic in which such a dispersion is blended and which is superior in emulsion stability.

There is provided a dispersion that has an extremely stable dispersibility, in which, by blending an ultraviolet light scattering agent coated with an inorganic oxide, aggregation of the coated ultraviolet light scattering agent is suppressed. There is also provided a cosmetic which, through use of an ultraviolet light scattering agent coated with the inorganic oxide and dispersant, or through use of the above dispersion, has a higher effect of ultraviolet light protection with an emulsion stability superior to that of a conventional product, as well as an improved use of feeling and safety.

### BACKGROUND OF THE INVENTION

1. Field of the Invention

The present invention relates to a dispersion (dispersed composition) used in a cosmetic for ultraviolet light protection such as prevention of sunburn, and more particularly, to a dispersion containing an ultraviolet light scattering agent coated with an inorganic oxide, and a dispersant. The present invention also relates to a cosmetic (cosmetic composition) and the like, containing the dispersion which suppresses aggregation of the ultraviolet light scattering agent to improve the effect in ultraviolet light protection.

According to the present invention, it is possible to suppress aggregation of the ultraviolet light scattering agent to be mixed, while it is also possible to disperse the ultraviolet light scattering agent uniformly in the dispersant and further to maintain the dispersed state for a prolonged time. Moreover, when the dispersion is used for a cosmetic, such a cosmetic can be provided, which is superior in its effect in ultraviolet light protection or the like.

2. Related Art

Recently, the adverse effects of ultraviolet light on the human body have generally been recognized, such that the protection against ultraviolet light has been of increasing importance. It is also well-known that the amount of ultraviolet light is increasing more and more every year due to the destruction of the ozone layer by freon (flon) gas, and the like. In order to prevent such adverse effects, a variety of products having protective effects against ultraviolet light (ultraviolet light protective effect) are currently being eveloped. This trend is likely to continue in the future. A rather easy way to exhibit high protective effects against ultraviolet light is to use larger proportions of ultraviolet light protective agent in the composition of a product. Ultraviolet light protecting products containing ultraviolet light scattering agents, are able to optimally shield the user from ultraviolet light with the agents being evenly scattered, and dispersed within the product itself. When ultraviolet light scattering agents are dispersed in a cosmetic, such as an emulsified composition for a cosmetic or the like, a dispersant such as a carboxy vinyl polymer, an alkyl modified carboxy polymer, or the like, is generally used. However, the ultraviolet light scattering agent aggregates either instantly or with a certain lapse of time, by an interaction with these dispersants, such that the ultraviolet light scattering effect (ultraviolet light protective effect) cannot be sufficiently exhibited. In particular, inultrafine (very finely pulverized) titanium oxide or ultrafine zinc oxide, exhibiting high ultraviolet light scattering effect, aggregation occurs readily because of the high surface activity of the primary particles. For this reason, such a method is required, which improves the ultraviolet light protective effect of the ultraviolet light scattering agent mixed in a cosmetic and achieves dispersion stability (stable dispersibility) thereof.

As a method for dispersion stabilization usually employed in the field of cosmetics, an anionic polymer material is used for gelation thereof to achieve the desired dispersion stabilization. However, in case that an inorganic powder (particle), such as titanium oxide, zinc oxide or the like, is mixed in a composition (cosmetic), sufficient dispersion (dispersibility)cannot be achieved by the above method. Also, in case that a nonionic polymer, such as cellulose type polymers or the like, is used, the yield point of the gel is extremely low, due to its structure, such that no sufficient dispersion stabilization can be obtained. Additionally, inorganic powder for cosmetics is usually a metal oxide or a complex oxide and is of a high specific gravity, such that, when the powder is used in a formulation of aqueous type, sufficient dispersion stability can be obtained unless some or other specific methods are used. Moreover, inorganic powders have an isoelectric point which means that aggregation can occur readily by inclusion of an electrolyte. Therefore, a dispersant such as a carboxyvinylpolymer or the like, which has been used in a system without powder, is difficult to use. Consequently, a gel structure created with the use of a surfactant (surface active agent), such as soap or the like, or an inorganic thickener, such as bentonite or the like, is used to achieve a stable dispersion in a conventional (prior) method. In such case, however, the gel strength is so difficult to adjust that it is difficult to produce a dispersion having fluidity, that is, a dispersion exhibiting acceptable dispersion stability.

Up to now, many proposals have been made to suppress aggregation of the ultraviolet light scattering agent in the dispersion, in case the ultraviolet light scattering agent is dispersed in the dispersant (for example, see Patent Reference 1 and the like). However, there is a need to further suppress the aggregation in the dispersion of ultraviolet light scattering agent and further improve the ultraviolet light protective effect.

For example, it has been reported that, by containing an aqueous (water) dispersion of the ultraviolet light scattering agent and polyacrylic amide, a cosmetic can be provided which is appreciably improved in dispersibility of the ultraviolet light scattering agent in the aqueous dispersion, and in the ultraviolet light protective effect (see Patent Reference 1). However, even though, in the reported cosmetic containing an aqueous dispersion of the ultraviolet light scattering agent and polyacrylic amide, aggregation of the ultraviolet light scattering agent in the polyacrylic amide has been improved to a certain extent, aggregation of the ultraviolet light scattering agent with polyacrylic amide occurs due to contact of the surface of the ultraviolet light scattering agent with a polyacrylic amide in the aqueous dispersion. That is, the reported cosmetic suffers from a problem that not only the effect of the ultraviolet light scattering agent is insufficient, but the emulsified composition is poor in stability, because the ultraviolet light scattering agent is not in a homogeneous mono-dispersed or substantially mono-dispersed state.

Under these circumstances, it is desirable to develop a dispersion in which aggregation of the ultraviolet light scattering agent is suppressed, and which is superior in dispersion stability of the ultraviolet light scattering agent as compared with prior art, that is, in case the ultraviolet light scattering agent is dispersed in the dispersant, a dispersion in which the ultraviolet light scattering agent is homogeneously dispersed in the dispersant, and is maintained in this dispersed state for a prolonged time, as well as a cosmetic, which contains such dispersion and has a high ultraviolet light protective effect and an excellent emulsion stability, and further which is superior in smooth feeling and safety.

The aforementioned Patent Reference 1 is Japanese Patent Kokai Publication JP-A-2002-326906.

### SUMMARY OF THE DISCLOSURE

In view of the above, it is an object of the present invention to provide a dispersion for a cosmetic which, even if the ultraviolet light scattering agent is dispersed in the dispersant, is extremely superior in dispersion stability, and a cosmetic containing this dispersion, and which has a high ultraviolet light protective effect, an excellent emulsion stability as compared to prior products, and further which is superior in smooth feeling and safety, and the like.

As a result of perseverant research towards achieving the above objective, the present inventor has found that, when an ultraviolet light scattering agent coated with an inorganic oxide (coated ultraviolet light scattering agent) is mixed with a dispersant, such a dispersion superior in dispersion stability can be prepared in which aggregation of the ultraviolet light scattering agent can be suppressed, while the coated ultraviolet light scattering agent can be homogeneously dispersed in the dispersion, and in which the dispersed state can be maintained for a prolonged time. The present inventor has also found that, with a cosmetic containing the aforementioned coated ultraviolet light scattering agent and the dispersant, or with a cosmetic containing the aforementioned dispersion containing the coated ultraviolet light scattering agent and the dispersant, it is possible to suppress the aggregation of the ultraviolet light scattering agent. It has been also found that, with the so prepared cosmetic, an ultraviolet light absorbing agent (absorber) can further be easily added to achieve (obtain) a high ultraviolet light protective effect, because the surface activity of the ultraviolet light scattering agent in an uncoated state is suppressed by using the aforementioned coated ultraviolet light scattering agent. Additionally, the cosmetic is superior in emulsion stability, smooth feeling and the like as compared to the prior products. A variety of these findings have led to the completion of the present invention.

According to a first aspect of the present invention, there is provided a dispersion comprising an ultraviolet light scattering agent coated with an inorganic oxide, and a dispersant, sometimes referred to as “the dispersion of the present invention”.

In a preferred embodiment of the present invention, the ultraviolet light scattering agent comprised in the dispersion is at least one selected from the group consisting of ultrafine titanium dioxide, ultrafine zinc oxide, ultrafine iron oxide, ultrafine cerium oxide, and ultrafine cerium phosphate-titanium phosphate complex, so that it is possible to prepare the coated ultraviolet light scattering agent having still higher dispersion stability and hence to prepare a dispersion having more excellent dispersion stability. Moreover, the inorganic oxide used for coating the ultraviolet light scattering agent can be selected from the group consisting of silica, alumina and zirconium oxide, so that it is possible to prepare the coated ultraviolet light scattering agent having still higher dispersion stability and hence to prepare a dispersion having more excellent dispersion stability.

In another preferred embodiment, the dispersant comprised in the dispersion of the present invention is a water-soluble polymer, and more preferably, at least one selected from the group consisting of polyvinyl alcohol, polyvinyl methyl ether, polyvinylpyrrolidone, poly(acrylamide), alkyl modified carboxyvinylpolymer, carboxyvinylpolymer, acrylic acid type copolymer, copolymer of polyvinylpyrrolidone and α-olefin, polyvinylpyrrolidone-vinyl acetate copolymer, copolymer of pyrrolidone-dimethyl aminoethyl methacrylate polymer, ethyl acrylate acryl copolymer, cross-linked polymer of alkyl vinyl ether and maleic anhydride, styrene-acrylic acid copolymer, vinyl naphthalene-maleic acid copolymer, diallyl dimethyl ammonium chloride acrylic acid-acrylamide copolymer, acrylamide-acrylic amide-2-methylpropane sulfonate, gums, cellulose derivatives, acrylamide type emulsion thickener, and acrylic acid type emulsion thickener, so that it is possible to prepare (obtain) a dispersion having more excellent dispersion stability.

According to another aspect of the present invention, there is provided a cosmetic, comprising of an ultraviolet light scattering agent coated with an inorganic oxide and a dispersant, or a cosmetic comprising the ultraviolet light scattering agent coated with the inorganic oxide and the dispersant as a dispersion, that is, a cosmetic containing the above defined dispersion. This cosmetic is sometimes referred to as “the cosmetic of the present invention”.

In a preferred embodiment of the present invention, the ultraviolet light scattering agent comprised in the cosmetic is at least one selected from the group consisting of ultrafine titanium dioxide, ultrafine zinc oxide, ultrafine iron oxide, ultrafine cerium oxide, and ultrafine cerium phosphate-titanium phosphate complex, so that it is possible to prepare (obtain) a cosmetic having more excellent emulsion stability. The inorganic oxide used for coating the ultraviolet light scattering agent may be selected from the group consisting of silica, alumina, and zirconium oxide, so that it is possible to prepare (obtain) a cosmetic having more excellent emulsion stability.

In another preferred embodiment, the dispersant comprised in the cosmetic of the present invention is a water-soluble polymer, and more preferably, at least one selected from the group consisting of polyvinyl alcohol, polyvinyl methyl ether, polyvinylpyrrolidone, poly(acrylamide), alkyl modified carboxyvinylpolymer, carboxyvinylpolymer, acrylic acid type copolymer, copolymer of polyvinylpyrrolidone and α-olefin, polyvinylpyrrolidone-vinyl acetate copolymer, copolymer of pyrrolidone-dimethyl aminoethyl methacrylate polymer, ethyl acrylate acryl copolymer, cross-linked polymer of alkyl vinyl ether and maleic anhydride, styrene-acrylic acid copolymer, vinyl naphthalene-maleic acid copolymer, diallyl dimethyl ammonium chloride acrylic acid-acrylamide copolymer, acrylamide-acrylic amide-2-methylpropane sulfonate, gums, cellulose derivatives, acrylamide type emulsion thickener, and acrylic acid type emulsion thickener, so that it is possible to prepare a cosmetic having more excellent emulsion stability.

In one embodiment of the present invention, the coated ultraviolet light scattering agent described above is preferably contained in an amount of 0.1 to 40 wt %, calculated as pure ultraviolet light scattering agent (non-coated), while the dispersant is preferably contained in an amount of 0.1 to 60 wt %, both ratios are based on the total weight of the cosmetic.

In another embodiment, the cosmetic of the present invention may be added by an additional amount of ultraviolet light absorbing agent to realize a higher ultraviolet light protective effect.

In yet another embodiment, the cosmetic of the present invention is used most preferably as a cosmetic of an aqueous solution type, solubilized type, emulsion type, dispersed powder type, water-oil bilayer type, or water-oil-powder type.

### PREFERRED EMBODIMENT OF THE INVENTION

The present invention is now explained with reference to the following preferred embodiments. The present invention includes a dispersion containing an ultraviolet light scattering agent coated with an inorganic oxide (coated ultraviolet light scattering agent) and a dispersant, a cosmetic containing an ultraviolet light scattering agent coated with an inorganic oxide (coated ultraviolet light scattering agent) and a dispersant, a cosmetic containing the aforementioned dispersion, and the like. Although the following description centers on the dispersion, it is merely an illustration and is not intended to limit the invention.

In describing the present invention, it must be noted that, as used in this description and the appended claims, the singular forms “a”, “an” and “the” include plural reference unless the content clearly dictates otherwise. Thus, for example, “an ultravioet light scattering agent” may include a mixture of two or more such agents, and the like.

(Dispersion of the Present Invention)

The dispersion of the present invention contains an ultraviolet light scattering agent coated with an inorganic oxide (termed below a coated ultraviolet light scattering agent) and a dispersant. The coated ultraviolet light scattering agent described above is not aggregated in the aforementioned dispersant, such that the coated ultraviolet light scattering agent can be homogeneously dispersed with excellent stability in the aforementioned dispersant. The dispersion of the present invention is extremely superior in dispersion stability as a dispersion for a cosmetic.

According to the present invention, there is no limitation to the ultraviolet light scattering agent coated with an inorganic oxide. For example, metal oxides, such as ultrafine titanium dioxide, ultrafine zinc oxide, ultrafine iron oxide, and ultrafine cerium oxide, complex oxides, such as ultrafine cerium phosphate-titanium phosphate complex , and the like, may be used. It is preferred to make selection from the group consisting of ultrafine titanium dioxide, ultrafine zinc oxide, ultrafine iron oxide, ultrafine cerium oxide, and ultrafine cerium phosphate-titanium phosphate complex. More preferably, as the ultrafine cerium phosphate-titanium phosphate complex described above , the complex represented by the general formula of CeₓTi₁₋ₓP₂O₇, where 0≦x≦1, is used. Concretely, for example, selection may be made from Ce_{0.95}Ti_{0.05}P₂O₇, Ce_{0.1}Ti_{0.90}P₂O₇, Ce_{0.05}Ti_{0.95}P₂O₇, Ce_{0.05}Ti_{0.5}P₂O₇, Ce_{0.07}Ti_{0.93}P₂O₇ and the like.

In the present invention ,the term “ultrafine” refers to particle having a particle size of 0.02 to 0.08 μm as an average (mean) particle size of the primary particle.

There is no particular limitation to the inorganic oxide(s) used as a coating substance (material) in the present invention. Preferably, the inorganic oxide(s) described above is selected from the group consisting of silica (silicon oxide), alumina (aluminum oxide), zirconium oxide and iron oxide. Inorganic hydroxide (s), such as iron hydroxide or the like, may also be used. One or more kinds of these inorganic oxides and hydroxides may also be used in combination.

In the present invention, the coating amount of the aforementioned inorganic oxides differs depending on the sort or the specific surface area of the ultraviolet light scattering agent. The coating amount of the inorganic oxides is preferably 2 to 40 wt % or so, and more preferably 5 to 30 wt % or so based on the amount of the ultraviolet light scattering agent prior to being coated, that is, the ultraviolet light scattering agent yet-to be coated.

There is no limitation to the method for coating the aforementioned inorganic oxide(s), such that the coating may be carried out by any prior method. For example, in case of coating silica, a plasma CVD method, a method consisting of spraying of ethyl silicate into a spiraling turbulent stream of an ultraviolet light scattering agent followed by direct current plasma jet processing, a method consisting of coating silica by reaction using a water-soluble silica salt and a reducing agent (reductant), and the like may be used. On the other hand, in case of coating alumina, a method consisting of coating a water-soluble aluminium salt by the hydrolysis followed by burning, or the like may be used.

In the so produced coated ultraviolet light scattering agent, a thin film of an inorganic oxide is formed on the surface of the ultraviolet light scattering agent. In such case, the film thickness of the thin film may be on the order of 0.1 to 10 nm on the average. The thin film of the aforementioned inorganic oxide, thus coating the ultraviolet light scattering agent, more specifically its surface, completely prohibits contact between the ultraviolet light scattering agent and the dispersant, so that it is possible to prevent or suppress aggregation of the ultraviolet light scattering agent due to the dispersant, to prevent or suppress destruction of an emulsion when the dispersion is used as an emulsified composition as well as to prevent a form of the emulsion from being changed to a hard gel.

Also, in the dispersion of the present invention, one or more kinds of the coated ultraviolet light scattering agents described above may be used for the dispersant within the scope of the present invention.

In the present invention, as the dispersant, preferably a water-soluble polymer may be selected. The water-soluble polymer are exemplified by, for example, polyvinyl alcohol, polyvinyl methyl ether, polyvinylpyrrolidone, poly(acrylamide), alkyl modified carboxyvinylpolymer, such as acrylic acid alkyl methacrylate copolymer, carboxyvinylpolymer, such as alkyl acrylate copolymer, acrylic acid type copolymer (for example, acrylates/cetheth-20 itaconate copolymer, acrylates/cetheth-20 methacrylate copolymer, acryates/steareth-50 acrylate copolymer, acrylates/steareth-20 itaconate copolymer, and acrylates/steareth-20 methacrylate copymer, entered in International Cosmetic Ingredient Dictionary), copolymer of polyvinylpyrrolidone and α-olefins, such as polyvinylpyrrolidone/eicosane copolymer, and polyvinylpyrrolidone/hexadecane copolymer, vinylpyrrolidone-vinyl acetate copolymer, copolymer of pyrrolidone-dimethyl aminoethyl methacrylate polymer, ethyl acrylate acryl copolymer, cross-linked polymer of alkyl vinylether and maleic anhydride, styrene-acrylic acid copolymer, vinyl naphthalene-maleic acid copolymer, diallyl dimethyl ammonium chloride acrylic acid-acrylamide copolymer, acrylamide-acrylic amide-2-methylpropane sulfonate, gums, such as acacia gum, benzoin gum, damar gum, guaiac gum, saponine, succinoglycan, karaya gum, tragacanthgum, carobbean gum, quince seed, estergum, agar-agar, casein, native gellan gum, xanthan gum, roast bean gum, carageenan, chitin, pullulan, guardlane (curdlan), dextran, gelatin, and pectin, and cellulose derivatives, such as methylcellulose, ethylcellulose, sodium carboxymethyl cellulose, hydroxyethyl cellulose, hydroxypropyl cellulose, promulgen, sodiumalginate, sodiumpectinate, starch sodium gluconate, propylene glycol alginate ester, and lignosulfate salt. Moreover, as the aforementioned water-soluble polymer, emulsion type of water-soluble polymer thickener, such as acrylamide type emulsion thickener and acrylic acid type emulsion thickener, may be used. Specifically, of these, alkyl modified carboxyvinylpolymer is preferably used.

As the water-soluble polymers described above, they may be purchased from any source in the market. For example, the Sepigel series (manufactured by SEPPIC Co.) and the like, as polyacrylic amide, CARBOPOL 1342, PEMULEN TR-1, PEMULEN TR-2 (manufactured by BF Goodrich Co.) and the like, as alkyl modified carboxyvinylpolymer, CARBOPOL 941 (manufactured by BF Goodrich Co.), which has a main chain of acrylic acid and comprises of a cross-linking of a acrylic sucrose or pentaerythrytol and the like, having a carboxyl group, High Bis Wako 105 (manufactured by WAKO PURE CHEMICALS INDUSTRIES LTD.) and the like, as a carboxyvinylpolymer, STRUCTURE 3000, STRUCTURE 2000 (manufactured by National Starch Co.), ACRYSOL 25, ACRYSOL 22, ACULYN 25, ACULYN 22 (manufactured by Rohm and Haas Co.), ANTIL 208 (manufactured by Gold Schmidt AG.) and the like, as an acrylic acid type copolymer, ANTARON V-220, ANTARON V-216 (manufactured by ISP (INTERNATIONAL SPECIALTY PRODUCTS) Co.) and the like, as a copolymer of a polyvinylpyrrolidone and α-olefin, Rubiscol VA (manufactured by BASF CORPORATION) and the like, as a vinyl pyrrolidone-vinyl acetate copolymer, Copolymer-937 (manufactured by ISP Co.), and the like, as a copolymer with vinyl pyrrolidone-dimethyl aminoethyl methacrylate polymer, Sepigel 305, Sepigel 501 (manufactured by SEPPIC Co.), Simagel 600 (manufactured by SEIWA KASEI Co., LTD) and the like, as an acrylamide type emulsion thickener, Simagel NS, Simagel EG, Simagel A and Simagel EPG (manufactured by SEIWAKASEI Co.,LTD) and the like, as an acrylic acid type emulsion thickener, and the like can be cited.

The aforementioned dispersant may be used singularly or as a mixture.

In the composition of the dispersion of the present invention, there is no particular limitation to the amounts of the aforementioned ultraviolet light scattering agent or the dispersant used. However, the aforementioned ultraviolet light scattering agent is preferably used in an amount of approximately 1 to 35 wt %, calculated as pure non-coated ultraviolet light scattering agent, while the aforementioned dispersant is preferably used in an amount of approximately 5 to 50 wt %. Furthermore, the concentration of the coated ultraviolet light scattering agent in the total composition of the dispersion is preferably 10 to 60 wt % or so and more preferably 30 to 50 wt % or so.

There is no particular limitation to the method of mixing and dispersing the coated ultraviolet light scattering agent described above in the dispersant described above, and any known methods may be used. For example, the dispersion can be prepared by a mixing/dispersing equipment, such as a sand grinding mill, a ball mill, a colloid mill, a homogenizer and the like.

In the dispersion containing the aforementioned ultraviolet light scattering agent and the aforementioned dispersant, as described above, the ultraviolet light scattering agent is maintained in a stable dispersed state in the dispersant by mechano-chemical processing. Accordingly, in the present invention, for the aforementioned dispersion, it is sufficient that the aforementioned ultraviolet light scattering agent and the aforementioned dispersant are contained in the composition, as a basic structure, while no surfactant needs to be contained in the composition. Therefore, there is no particular limitation to the production of a cosmetic containing the dispersion, such that, in case that the dispersion is applied to water, an emulsified composition, a gel composition, or the like, the dispersion may readily be dispersed with excellent stability.

The dispersion of the present invention may be further added by any humectant, such as 1,2-pentanediol, 1,2-hexanediol, 1,2-heptanediol, polyethylene glycol, propylene glycol, dipropylene glycol, 1,3-butylene glycol, hexylene glycol, glycerin, diglycerin, sorbitol, and the like.

It should be noted that a dispersion superior in dispersion stability may also be provided if white pigments of pigmentary grade used for cosmetics (with the same particle size as that of a pigment, or on the order of 0.1 to 0.2 μm as an average particle size), such as titanium dioxide, zinc oxide and iron oxide, or inorganic colorants, such as ultramarine blue and Berlin blue, or clay minerals, such as kaolin, talc, sericite, mica and magnesium carbonate, or incinerated pigments, such as cobalt titanate and titanium dioxide-iron oxide, or doped powder pigments, or organic pigments, such as Red 226, or lake pigments, such as Red 202, Yellow 5, and Blue 1, are coated, in place of the ultraviolet light scattering agent used in the present invention, with the inorganic oxides, to prepare a coated product, which then is dispersed in the dispersant. Moreover, the cosmetic containing such dispersion, or the cosmetic containing such coated product (coated product obtained on coating the aforementioned white pigment or the like with the inorganic oxide) and the dispersant, is superior in emulsion stability than prior products, and further being acceptable in smooth feeling and safety.

(Cosmetic of the Present Invention)

The cosmetic of the present invention is a cosmetic containing the aforementioned ultraviolet light scattering agent coated with the inorganic oxide, and the dispersant, or a cosmetic containing the aforementioned dispersion (dispersion of the present invention) That is, the cosmetic may be prepared by mixing each of the ultraviolet light scattering agent coated with the inorganic oxide and the dispersant, or by mixing the dispersion prepared by using the coated ultraviolet light scattering agent and the dispersant. Meanwhile, the ultraviolet light scattering agent coated with the inorganic oxide may be prepared as described above, while the dispersion may also be prepared as described above.

In the present invention, the cosmetic products of the present invention can be produced without any difficulties using existing technologies. Especially, technologies used to produce cosmetics with the ultraviolet light scattering agent are useful in making emulsions and the like and obtaining desired cosmetics.

In the cosmetic of the present invention, when the above-described coated ultraviolet light scattering agent is used, the amount of mixing of the coated ultraviolet light scattering agent described above and the dispersant described above may be optionally selected depending on the properties of the desired cosmetic. As for the amount of the coated ultraviolet light scattering agent described above, it is on the order preferably of 0.1 to 40 wt %, more preferably of 1.0 to 30 wt % and most preferably of 1.0 to 20 wt %, based on the total cosmetic composition. As for the dispersant described above, it is on the order preferably of 0.01 to 60 wt % and more preferably on the order of 0.05 to 20 wt % in order to provide a strong smooth feeling, specifically a strong water-off feeling in the gel composition and emulsified composition at an early stage of application thereof, and to moisturize, and to provide a dewy feeling as well as being transparent, and have a moderate powdery feeling after application to the skin, based on the total cosmetic composition. When the mixed amount (amount of use) is less than 0.01 wt %, difficulties are met in emulsification, and the precipitation of pigment or the like occurs, so that problems arise in stability, while the water-off feeling is undesirably lost. When the mixed amount exceeds 60 wt %, the emulsifying action and the emulsion stability cannot be expected to increase, while the cosmetic obtained does not have good extension on the skin nor affixture, while a feeing of worseness is produced therein, so that the effect of moisturizing or dewy feeling decreases.

In the cosmetic of the present invention, when the aforementioned dispersion (dispersion of the present invention) is used, the mixed amount of the aforementioned dispersion is arbitrarily selected in dependence upon the properties of the desired cosmetic. It is possible to mix the dispersion in an amount preferably of 2 to 60 wt % or so, more preferably 3 to 50 wt % or so and most preferably 5 to 40 wt % or so in the total cosmetic composition.

The cosmetic of the present invention may further be added by an ultraviolet light absorbing agent. For example, as the ultraviolet light absorbing agent, cinnamic acid type ultraviolet light absorbing agents, such as octyl cinnamate, ethyl-4-isopropyl cinnamate, methyl-2,5-diisopropyl cinnamate, ethyl-2,4-diisopropyl cinnamate, methyl-2,4-diisopropyl cinnamate, propyl-p-methoxy cinnamate, isopropyl-p-methoxy cinnamate, isoamyl-p-methoxy cinnamate, octyl-p-methoxy cinnamate, 2-ethoxyethyl-p-methoxy cinnamate, cyclohexyl-p-methoxy cinnamate, ethyl-α-cyano-β-phenyl cinnamate, 2-ethylhexyl-α-cyano-β-phenyl cinnamate, and glycerylmono-2-ethyl hexanoyl-diparamethoxy cinnamate, benzophenone type ultraviolet light absorbing agents, such as 2,4-dihydroxy benzophenone, 2,2′-dihydroxy-methoxy benzophenone, 2,2′-dihydroxy-4,4′-dimethoxy benzophenone, 2,2′,4,4′-tetrahydroxy benzophenone, 2-hydroxy-4-methoxy benzophenone, 2-hydroxy-4-methoxy 4′-methyl benzophenone, 2-hydroxy-4-methoxybenzophenone-5-sulfonate, 4-phenyl benzophenone, 2-ethylhexyl-4′-phenyl-benzophenone-2-carboxylate, 2-hydroxy-4-n-octoxy benzophenone, and 4-hydroxy-3-carboxy benzophenone, para-aminobenzoic acid type ultraviolet light absorbing agents, such as PABA monoglycerin ester, N,N-dipropoxy PABA ethyl ester, N,N-diethoxy PABA ethyl ester, N,N-dimethyl PABA ethyl ester, N,N-dimethyl PABA butyl ester, and N,N-dimethyl PABA methyl ester, salicylic acid type ultraviolet light absorbing agents, such as amino salicylate, menthyl salicylate, homomenthyl salicylate, octyl salicylate, phenyl salicylate, benzyl salicylate, and p-isopropanol phenyl salicylate, anthranylic acid type ultraviolet light absorbing agents, such as methyl anthranylate, 3-(4′-methylbenzylidene)-d-camphor, 3-benzylidene-d, 1-camphor, urocanic acid, urocanic acid ethyl ester, octyltriazone, 2-phenyl-5-methyl benzoxazole, 2-(2′-hydroxy-5′-methylphenyl) benzotriazole, and 4-methoxy-4′-t-butyl dibenzoyl methane, and the like can be cited. As for the mixed amounts of these ultraviolet light absorbing agents, a high ultraviolet light protective effect may be achieved (obtained) even with a small mixed amount because the ultraviolet light absorbing agent in the cosmetic is retained in an acceptable dispersed state.

The cosmetic of the present invention may be added by other ingredients routinely used for cosmetics, such as liquid fats, solid fats, liquid or solid fats, waxes, ester oils, hydrocarbon oils, silicone, lower alcohols, sterols, humectants, sequestering agents, neutralizers, pH adjusting agents, anti-oxidants, antibacterial agents, a variety of liquid extracts, medicaments and the like, in the range without impairing the object(s) of the present invention and the effect(s) obtained in the present invention.

As the aforementioned liquid fats, for example, linseed oil, camellia oil, macadamia nut oil, corn oil, mink oil, olive oil, avocado oil, sasanqua oil, castor oil, safflower oil, apricot kernel oil, cinnamon oil, jojoba oil, grape oil, sunflower oil, almond oil, rapeseed oil, sesame oil, wheat germ oil, rice germ oil, rice bran oil, cottonseed oil, soybean oil, peanut oil, tea seed oil, evening primrose oil, egg yolk oil, neatsfoot oil, liver oil, triglycerin, glycerin trioctanoate, glycerin triisopalmitate and the like, can be cited.

As the aforementioned solid fats, for example, cacao butter, beef tallow, mutton tallow, lard, horse fat, hardened oil, hardened castor oil, Japan wax, shea butter and the like, can be cited.

As the aforementioned liquid or solid fats, coconut oil, palm oil, palm kernel oil and the like, can be cited.

As the aforementioned waxes, bees wax, candelilla wax, cotton wax, carnauba wax, bayberry wax, Chinese wax, spermaceti wax, montan wax, bran wax, lanolin, reduced lanolin, hard lanolin, kapok wax, sugarcane wax, jojoba wax, shellac wax and the like, can be cited.

As the aforementioned ester oils, octanoic acid esters, such as cetyl octanoate, isooctanoic acid esters, such as glycerin tri-2-ethyl hexanoate, and pentaerythritol tetra-2-ethyl hexanoate, lauric acid esters, such as hexyl laurate, myristic acid esters, such as isopropyl myristate and octyldodecyl myristate, palmitic acid esters, such as octyl palmitate, stearic acid esters, such as isocetyl stearate, isostearic acid esters, such as isopropyl isostearate, isopalmitic acid esters, such as octyl isopalmitate, oleic acid esters, such as octyldedecyl oleate, adipic acid diesters, such as diisopropyl adipate, and sebacic acid diesters, such as diethyl sebacate, and diisostearyl malate, and the like, can be cited.

As the aforementioned hydrocarbon oils, liquid paraffin, ozokerite, squalane, squalene, pristane, paraffin, isoparaffin, ceresin, vaseline (petrolatum), microcrystalline wax, and the like, can be cited.

As the aforementioned silicones, linear silicones, such as dimethyl polysiloxane, methylphenyl polysiloxane and methyl hydrogen polysiloxane, and cyclic silicones, such as octamethyl cyclotetrasiloxane, decamethyl cyclopentasiloxane, and dodecamethyl cyclohexasiloxane, and the like, can be cited.

As the aforementioned lower alcohols, methanol, ethanol, propanol, isopropanol, and the like can be cited.

As the aforementioned sterols, cholesterol, sitosterol, phytosterol, lanosterol, and the like can be cited.

As the aforementioned humectants, 1,2-pentanediol, 1,2-hexanediol, 1,2-heptanediol, polyethylene glycol, propylene glycol, dipropylene glycol, 1,3-butylene glycol, hexylene glycol, glycerin, diglycerin, sorbitol, and the like can be cited.

As the aforementioned sequestering agents, alanine, sodium edetate salt, sodium polyphosphate, sodium metaphosphate, phosphoric acid, and the like can be cited.

As the aforementioned neutralizers, 2-amino-2-methyl-1-propanol, 2-amino-2-methyl-1,3-propanediol, potassium hydroxide, sodium hydroxide, amino acids, such as L-alguinine and L-lysine, triethanolamine, sodium carbonate, and the like can be cited.

As the aforementioned pH adjusting agents, lactic acid, citric acid, glycolic acid, succinic acid, tartaric acid, dl-malic acid, potassium carbonate, sodium hydrogen carbonate, ammonium hydrogen carbonate, and the like can be cited.

As the aforementioned anti-oxidants, ascorbic acid, α-tocopherol, dibutyl hydroxytoluene, butyl hydroxyanisole, and the like can be cited.

As the aforementioned antibacterial agents, benzoic acid, salicylic acid, carbolic acid, sorbic acid, paraoxy benzoate, parachloro metacresol, hexachlorophene, benzalkonium chloride, chlorhexidine chloride, trichloro calbanilide, sensitizing dyes, phenoxy ethanol, and the like can be cited.

As the variety of liquid extracts described above, houttuynia extract, phellodendron bark extract, sweet clover extract, white nettle extract, glycyrrhiza extract, peony root extract, saponaria extract, sponge gourd extract, cinchona extract, saxifrage extract, sophora root extract, nuphar extract, fennel extract, primrose extract, rose extract, rehmannia root extract, lemon extract, lithospermum root extract, aloe extract, calamus rhizome extract, eucalyptus extract, horsetail extract, sage extract, thyme extract, green tea extract, seaweed extract, cucumber extract, clove extract, raspberry extract, balm mint extract, ginseng extract, carrot extract, horse chestnut extract, peach extract, peach leaf extract, mulberry bark extract, cornflower extract, witch hazel extract, placental extract, thymus gland extact, silk extract, and the like can be cited.

As the aforementioned medicaments, vitamins, such as vitamin Aoil, retinol, retinolpalmitate, inocitol, pyridoxine hydrochloride, benzyl nocotinate, nicotinic acid amide, DL-α-tocopheryl nicotinate, ascorbic acid magnesium phosphate, vitamin D2 (ergocalciferol), d1-α-tocopherol, d1-δ-tocopherol, potassium diester ascorbate, d1-α-tocopheryl acetate, pantothenic acid and biotin, hormones, such as estradiol and ethinyl estradiol, amino acids, such as alguinine, aspartic acid, cystine, cysteine, methionine, serine, leucine and tryptophan, anti-inflammatory agents, such as allantoin, grycyrrhethinic acid and azulene, whitening agents, such as arbutin, astringents, such as zinc oxide and tannic acid, refrigerants, such as L-menthol and camphor, sulfur, lyzozyme chloride, pyridoxine chloride and γ-oryzanol, and the like can be cited. Furthermore, the aforementioned medicaments may be used not only in a free state but also in the form of a salt of an acid or a base if the medicament is of the salt-making type. Also, the medicaments having a carboxylic acid group may be used in the form of an ester.

In the cosmetic of the present invention, any suitable perfumes, colorants and the like may be used in the range without impairing the object(s) of the present invention and the effect(s) (such as emulsion stability or the like) obtained in the present invention.

There is no particular limitation to the agent form of the cosmetic of the present invention. That is, the cosmetic may be in any types, such as an aqueous solution type, solubilized type, emulsion type, powder dispersion type, water-oil bi-layer type, or water-oil-powder type and the like. For example, a skin care essence, under-makeup gel, foundation, hair styling gel, body gel, massage gel, wipe-off pack, sunscreen emulsion, sunscreen cream, hair styling agent, hair cream, hair rinse, hair dye, sunscreen gel, moisture gel, sunscreen lotion, suncum lotion and the like can be cited. Specifically, the cosmetic of the present invention is preferably used as a cosmetic of an aqueous solution type, solubilization type, emulsion type, powder dispersion type, water-oil bi-layer type, or water-oil-powder type.

This application is based on the Japanese Patent Application Serial No.2003-163893, field on Jun. 9, 2003, which is incorporated herein by reference in its entirety.

### EXAMPLES

The present invention is further explained with reference to the following Examples and Comparative Examples. However, the present invention is not limited to these Examples and Comparative Examples which are given merely for the sake of illustration.

### Example 1

### Preparation Example of Coated Ultraviolet Light Scattering Agent-1

Ultrafine titanium dioxide (TTO-55A: manufactured by Ishihara Sangyo Kaisha, LTD.) were coated with silica by a plasma CVD method to produce silica coated ultrafine titanium dioxide (coating amount of silica, 14.0%; average film thickness, 4.4 nm).

### Example 2

### Preparation Example of Coated Ultraviolet Light Scattering Agent-2

Ultrafine titanium dioxide (TTO-55A: manufactured by Ishihara Sangyo Kaisha, LTD.) were coated with silica by a reaction employing sodium silicate and a reducing agent to produce silica coated ultrafine titanium dioxide (coating amount of silica, 12.4%; average film thickness, 3.8 nm).

### Example 3

### Preparation Example of Coated Ultraviolet Light Scattering Agent-3

Silica coated ultrafine zinc oxide (coating amount of silica, 14.0%; average film thickness, 4.0 nm) were prepared in the same way as in Example 1 except using ultrafine zinc oxide (FINEX25: manufactured by SAKAI CHEMICAL INDUSTRY CO., LTD.) in place of ultrafine titanium dioxide (TTO-55A: manufactured by Ishihara Sangyo Kaisha, LTD.) in Example 1.

### Example 4

### Preparation Example of Coated Ultraviolet Light Scattering Agent-4

Alumina coated ultrafine zinc oxide (coating amount of alumina, 15.4%; average film thickness, 4.8 nm) were prepared by hydrolyzing sodium aluminate in the presence of ultrafine zinc oxide (FINEX50: manufactured by SAKAI CHEMICAL-INDUSTRY CO., LTD.) and subsequently burning the resulting product at 550° C. for five hours.

Further, in the above Examples 1 to 4, the coated inorganic oxides were determined by fluorescent X ray (method), while the thickness of the coated layer was calculated from a photo of a transmission electron microscope.

### Example 5

### Preparation Example of Dispersion-1

A dispersion was prepared according to the following method for production, based on the compositions of the following table 1.

(Method for Production)

1. To a purified water, an acrylic acid alkyl methacrylate copolymer (PEMULEN TR-2, manufactured by BF Goodrich Co.) was added and the resulting product was agitated with a stirrer for dissolution.

2. To the aqueous solution, obtained in 1, triethanolamine was added and thereby neutralized to produce a solution of the acrylic acid alkyl methacrylate copolymer.

3. The coated ultraviolet light scattering agent, obtained in Example 1, was accurately weighed so that the coated ultraviolet light scattering agent amounts to 2.5 wt %, calculated as pure ultraviolet light scattering agent, based on the total weight of the mixture of the dispersant component and other components. The so weighed out coated ultraviolet light scattering agent was added to the acrylic acid alkyl methacrylate copolymer obtained in 2 and dispersed sufficiently.

TABLE 1
Composition of the Dispersion (unit: wt %)

| components | | Amount |
|---|---|---|
| ultraviolet light | coated ultraviolet light | 2.5* |
| scattering agent | scattering agent obtained in | |
| | Example 1 | |
| dispersant | acrylic acid alkyl methacrylate | 0.2 |
| | copolymer** | |
| other components | Triethanolamine | 0.2 |
| | purified water | 99.6 |

*The coated ultraviolet light scattering agent, obtained in Example 1, was added additively so that the coated ultraviolet light scattering agent amounts to 2.5 wt %, calculated as pure ultraviolet light scattering agent (uncoated), based on the total weight of the mixture of the dispersent component and other components.
**PEMULEN TR-2 (manufactured by BF Goodrich Co.).

### Comparative Example

### Preparation Example of Dispersion-2

A dispersion was prepared (produced) in the same way as in Example 5 except using uncoated ultrafine titanium dioxide (TTO-55A manufactured by Ishihara Sangyo Kaisha, LTD.) in place of the coated ultraviolet light scattering agent prepared in Example 1.

### Example 6

### Evaluation of Ultraviolet Light Protective Effect and Dispersibility

The ultraviolet light protective effect of the dispersions obtained in Example 5 and in the Comparative Example was evaluated by determining SPF value by the following method. Additionally, SPF value of a solution of an acrylic acid alkyl methacrylate colopymer (PEMULEN TR-2, manufactured by BF Goodrich Co.), as a control, were also determined by the following method. The dispersibilities in the above-dispersions and the control were also evaluated respectively by visual observation.

(Measurement of SPF (Sun Protection Factor))

1. On a quartz plate with length of 100 mm, width of 100 mm and thickness of 3 mm, a transpore surgical tape, manufactured by 3M Co., was affixed, and on this tape an area 6.4 cm×6.4 cm (approxymately 40 cm²) for sample application was indicated.

2. On the above area for application, 0.08 g of the solution of the acrylic acid alkyl methacrylate colopymer (sample 1) was applied with a sponge puff at an amount of approximately 2.0 mg/cm², and allowed to stand for 15 minutes.

3. Using an SPF analyzer (SPF-290 Analyzer, manufactured by Optometrics Co.), a measurement light with an irradiation area of 16 mm in diameter was irradiated on the surface of the quartz plate where the solution of the acrylic acid alkyl methacrylate colopymer (sample 1) was applied, and measured at 9 different spots. The SPF value was derived from the mean value of the 9 measured values.

4. For each sample, the above operations from 1 to 3 were repeated thrice to determine a mean value.

Additionally, the SPF value was determined for the dispersion (sample 2) obtained in Comparative Example and the dispersion (sample 3) obtained in Example 5 in a similar manner as above. These results of the evaluation are shown in the following Table 2.

TABLE 2
Results of Evaluation

| | | | Evaluations of | |
|---|---|---|---|---|
| | | SPF | dispersibility by | evalua- |
| samples | | values | visual observations | tions |
| 1 | solution of acrylic | 1.0 | — | — |
| | acid alkyl | | | |
| | methacrylate | | | |
| | copolymer* | | | |
| 2 | dispersion obtained in | 1.0 | small agglomerations of | X |
| | Comparative Example | | particles of | |
| | | | approximately 0.5 mm | |
| | | | were seen by visual | |
| | | | observation | |
| 3 | dispersion obtained in | 8.1 | there were no particles | ⊚ |
| | Example 5 | | seen by visual | |
| | | | observation; in case of | |
| | | | spreading on a glass | |
| | | | plate, there were no | |
| | | | agglomerated particles | |

*PEMULEN TR-2 (manufactured by BF Goodrich Co.)

(Results of Evaluation)

The SPF value of the dispersion, obtained in Example 5, is much higher than that of the dispersion obtained in the Comparative Example. This is attributed to the fact that, in the silica-coated ultrafine titanium dioxides, contained in the dispersion obtained in Example 5, the ultrafine titanium dioxides are coated with the coating film homogeneously and completely on the surface thereof.

Also, the results of evaluation of dispersibility by visual observation indicated that, with the dispersion obtained in the Comparative Example, ultrafine titanium dioxides, were seen to be aggregated with a size of approximately 0.5 mm (particles), and after the dispersion was allowed to stand for a certain amount of time, phase separation (precipitation of particles) occurred. Conversely, with the dispersion obtained in Example 5, no ultrafine titanium dioxide (particle) agglomeration could be seen with the naked eye. Also, when the dispersion was spread on the glass plate, no ultrafine titanium dioxide agglomeration was presented.

It maybe seen from above that, with the dispersion of the present invention, the ultraviolet light scattering agent is dispersed in the dispersant homogeneously and with good stability. From this, it is apparent that such dispersion is superior in dispersion stability of the ultraviolet light scattering agent. Therefore, the present invention can provide a dispersion, which is extremely superior in dispersion stability of the ultraviolet light scattering agent because the ultraviolet light scattering agent can be dispersed in the dispersant homogeneously and with good stability.

### Example 7

### Preparation Example of Cosmetic-1

A skin care essence was produced according to the following method for production, based on the composition of the following Table 3.

(Method for Production)

1. The following components of phase A were heated to solubilize and dispersed (mixed).

2. To the mixture obtained in 1, the following components of phase B were added to neutralize. Subsequently, the mixture thus obtained was cooled while stirring to obtain a desired cosmetic (skin care essence).

TABLE 3
Composition of Cosmetic (skin care essence)
(unit: parts by weight)

| components | | Amount |
|---|---|---|
| phase A | silica coated ultrafine titanium dioxide | 10.0 |
| | obtained in Example 1 | |
| | 1,3-butylene glycol | 10.0 |
| | sodium chondroitin sulfate | 0.2 |
| | methylparaben | 0.2 |
| | acrylic acid alkyl methacrylate copolymer* | 0.2 |
| | Polyoxyethylene methyl polysiloxane copolymer | 0.5 |
| | POE (30) POP (6) decyltetradecylether | 0.5 |
| | eucalypt extract | 1.0 |
| | alkylol hydrolyzed collagen aminopropane diol | 0.2 |
| | salt | |
| | purified water | 60.0 |
| phase B | triethanolamine | 0.3 |
| | purified water | 16.9 |

*PEMULEN TR-2 (manufactured by BF Goodrich Co.)

(Evaluation of Cosmetic)

After the cosmetic (skin care essence) obtained was allowed to stand at 40° C. for three months, the ultraviolet light protective effect thereof was evaluated by determining the SPF value by a similar manner as the method for measuring the SPF value in Example 6.

As a result, the SPF value thereof was found to be 28.0.

### Example 8

### Preparation Example of Cosmetic-2

A sunscreen lotion was prepared (produced) according to the following method for production, based on the composition of Table 4.

(Method for Production)

1. The following components of phase A were heated to solubilize and dispersed.

2. Subsequently, to the mixture obtained in 1, the mixture of the following components of phase B was added, and further the following component of phase C was added. The resulting mixture was stirred to produce (obtain) a desired cosmetic (sunscreen lotion).

TABLE 4
Composition of Cosmetic (sunscreen lotion)
(unit: parts by weight)

| components | | Amount |
|---|---|---|
| phase A | acrylic acid alkyl methacrylate copolymer* | 1.74 |
| | Triethanolamine | 0.2 |
| | purified water | 53.85 |
| | 4-tert-butyl 4′-methoxy-dibenzoylmethane | 1.0 |
| | 2-ethylhexyl paramethoxycinnamate | 7.0 |
| | 2-octyldodecyl pivalate | 3.0 |
| phase B | propylene glycol dispersion of silica coated | 12.5 |
| | ultrafine titanium dioxide obtained in Example 1** | |
| | silica beads | 0.8 |
| phase C | ethanol | 20.0 |

*2% aqueous solution of PEMULEN TR-2 (manufactured by BF Goodrich Co.)
**The concentration of silica coated ultrafine titanium dioxide obtained in Example 1 is 40 wt %.

(Evaluation of Cosmetic)

After the cosmetic (sunscreen lotion) obtained was allowed to stand at 40° C. for three months, this cosmetic was checked as to whether or not any discoloration of the ultraviolet light absorbing agent occured and as to whether or not separation and/or gelation occurred. The ultraviolet light protective effect of this cosmetic was also evaluated by determining the SPF value by a similar manner as the method for measuring the SPF value in Example 6.

As a result, it was found that neither discoloration of the ultraviolet light absorbing agent, nor the separation and/or gelation in the cosmetic was exhibited, with the system being in a stable state. The SPF value thereof was found to be 45.3.

### Example 9

### Preparation Example of Cosmetic-3

Based on the composition of the following Table 5, a moisturizer was prepared according to the following method for production.

(Method for Production)

1. To the following component of phase A, each component of the following components of phase B was added, and the resulting mixture was dissolved and dispersed at 90° C.

2. Subsequently, as the mixture obtained in 1 was cooled while stirring, the following component of phase C was added to the resulting product (mixture) to adjust the pH value to 8.0. The resulting product was cooled to 50° C. to produce a desired cosmetic (moisturizer).

TABLE 5
Composition of Cosmetic (moisturizer) (unit: parts by weight)

| components | | Amount |
|---|---|---|
| phase A | purified water | to 100.0 |
| phase B | propylene glycol | 6.0 |
| | polyethylene glycol 400 | 3.0 |
| | magnesium ascorbate | 0.5 |
| | POE sorbitane monolaurate | 0.4 |
| | agar | 1.0 |
| | native gellan gum | 0.4 |
| | trimethylglycine | 1.0 |
| | 1,3-butylene glycol dispersion of silica | 33.3 |
| | coated ultrafine zinc oxide obtained in Example 3* | |
| phase C | pH adjusting agent | the right |
| | | amount |

*The concentration of silica coated ultrafine zinc oxide obtained in Example 3 is 30 wt %.

(Evaluation of Cosmetic)

After the cosmetic (moisturizer) obtained was allowed to stand at 40° C. for three months, this cosmetic was checked as to whether or not the cosmetic was affected in stability such as separation, gelation, and the like thereof. The ultraviolet light protective effect of this cosmetic was also evaluated by determining the SPF value by a similar manner as the method for measuring the SPF value in Example 6.

As a result, it was found that no changes in stability, such as separation, and gelation, and the like occurred in the cosmetic, with the system being in a stable state. The SPF value thereof was found to be 13.3.

### Example 10

### Preparation Example of Cosmetic-4

Based on the composition of the following Table 6, a cream was prepared according to the following method for production.

(Method for Production)

1. The following components of phase A were dissolved at 80° C.

2. The components of phase B which were mixed through heating in advance, were added to the mixture obtained in 1, and the resulting product (mixture) was emulsified with stirring.

3. Subsequently, the mixture obtained in 2 was cooled to 40° C. to produce a desired cosmetic (cream).

TABLE 6
Composition of Cosmetic (cream) (unit: parts by weight)

| components | | Amount |
|---|---|---|
| phase A | polyvinyl pyrrolidone/eicosene copolymer* | 2.0 |
| | squalane | 2.0 |
| | dimethyl polysiloxane | 3.0 |
| | tri-2-glyceryl ethyl hexanoate | 3.0 |
| Phase B | acrylic acid methacrylic acid copolymer** | 0.2 |
| | purified water | 62.5 |
| | sodium hydroxide | 0.1 |
| | 1,3-butyleneglycol | 7.0 |
| | methylparaben | 0.2 |
| | silica coated ultrafine titanium dioxide | 15.0 |
| | obtained in Example 2 | |
| | alumina coated ultrafine zinc oxide obtained in | 5.0 |
| | Example 4 | |

*Antaron V-220 (manufactured by ISP Co.)

(Evaluation of Cosmetic)

After the cosmetic (cream) obtained was allowed to stand at 50° C. for one month, this cosmetic was checked as to whether or not the cosmetic was affected in stability such as separation, gelation and the like in the cosmetic. The ultraviolet light protective effect of this cosmetic was also evaluated by determining the SPF value by a similar manner as the method for measuring the SPF value in Example

As a result, it was found that no changes in stability, such as separation, and gelation, and the like occurred in the cosmetic, with the system being in a stable state. The SPF value thereof was found to be 37.1.

### Example 11

### Preparation Example of Cosmetic-5

Based on the composition of the following Table 7, a foundation was prepared according to the following method for production.

(Method for Production)

1. The following components of phase A were dissolved and dispersed on heating at 90° C., and the mixture thus obtained was kept at 70° C.

2. The following components of phase B were subjected to three-roller mill processing, and the mixture thus obtained was kept at 70° C.

3. The mixture obtained in 1 and the mixture obtained in 2 were mixed together and emulsified in a homogenizer, and subsequently, the mixture thus obtained was cooled to 40° C. to produce a desired cosmetic (foundation).

TABLE 7
Composition of Cosmetic (foundation) (unit: parts by weight)

| components | | Amount |
|---|---|---|
| phase A | purified water | 73.2 |
| | 1,3-buthylene glycol | 5.0 |
| | glycerine | 5.0 |
| | guar gum | 0.4 |
| | agar | 0.9 |
| | silica coated ultrafine titanium dioxide | 5.7 |
| | obtained in Example 2 | |
| | methylparabene | 0.2 |
| phase B | silicone treated talc | 5.0 |
| | silicone treated sericite | 2.0 |
| | silicone treated titanium dioxide* | 15.0 |
| | amino acid treated red iron oxide | 1.5 |
| | amino acid treated yellow iron oxide | 3.0 |
| | amino acid treated black iron oxide | 0.5 |
| | decamethyl cyclopentasiloxane | 10.0 |
| | polyether modified silicone | 1.0 |
| | methylphenyl polysiloxane | 10.0 |
| | dimethyl polysiloxane | 8.0 |

*pigmentary grade

(Evaluation of Cosmetic)

After the cosmetic (foundation) obtained was allowed to stand at 50° C. for one month, this cosmetic was checked as to whether or not the cosmetic was affected in stability such as separation, gelation and the like in the cosmetic. The ultraviolet light protective effect of this cosmetic was also evaluated by determining the SPF value by a similar manner as the method for measuring the SPF value in Example 6.

As a result, it was found that no changes in stability, such as separation, and gelation, and the like occurred in the cosmetic, with the system being in a stable state. The SPF value thereof was found to be 15.8.

From the foregoing, it is clear that when, in the cosmetic of the present invention, the ultraviolet light scattering agent is dispersed in a dispersant, aggregation of the ultraviolet light scattering agent can be suppressed, and the ultraviolet light scattering agent can be dispersed in the dispersant homogeneously and with good stability can for a prolonged time with a dispersant. Moreover, such cosmetic can be produced with a simple process. Therefore, cosmetics having a high ultraviolet light protective effect and emulsion stability superior to that of the existing cosmetic products and which is excellent in application felling (smooth feeling) and safety can be provided according to the present invention.

The meritorious effects of the present invention are summarized as follows.

The present invention can provide a dispersion containing an ultraviolet light scattering agent blended (dispersed) in the dispersant, wherein aggregation of the ultraviolet light scattering agent is suppressed and which is extremely excellent in dispersion stability.

Moreover, a cosmetic having a high ultraviolet light productive effect and an excellent smooth feeling or the like can readily be produced with a simple process, using the aforementioned ultraviolet light scattering agent coated with the inorganic oxide, and the aforementioned dispersant, or using the aforementioned dispersion. Therefore, the present invention is industrially extremely useful especially in the field of cosmetics.

It should be noted that other objects, features and aspects of the present invention will become apparent in the entire disclosure and that modifications may be done without departing the gist and scope of the present invention as disclosed herein and claimed as appended herewith.

Also it should be noted that any combination of the disclosed and/or claimed elements, matters and/or items may fall under the modifications aforementioned.

What is claimed is:

1. A dispersion comprising an ultraviolet light scattering agent coated with an inorganic oxide, and a dispersant.

2. The dispersion of claim 1, wherein said ultraviolet light scattering agent is selected from the group consisting of ultrafine titanium dioxide, ultrafine zinc oxide, ultrafine iron oxide, ultrafine cerium oxide, and ultrafine cerium phosphate-titanium phosphate complex.

3. The dispersion of claim 1, wherein said inorganic oxide is selected from the group consisting of silica, alumina and zirconium oxide.

4. The dispersion of claim 1, wherein said dispersant is a water-soluble polymer.

5. The dispersion of claim 4, wherein said water-soluble polymer is at least one selected from the group consisting of polyvinyl alcohol, polyvinyl methyl ether, polyvinylpyrrolidone, poly(acrylamide), alkyl modified carboxyvinylpolymer, carboxyvinylpolymer, acrylic acid type copolymer, copolymer of polyvinylpyrrolidone and α-olefin, polyvinylpyrrolidone-vinyl acetate copolymer, copolymer of pyrrolidone dimethyl aminoethyl methacrylate polymer, ethyl acrylate acryl copolymer, cross-linked polymer of alkyl vinyl ether and maleic anhydride, styrene-acrylic acid copolymer, vinyl naphthalene-maleic acid copolymer, diallyl dimethyl ammonium chloride acrylic acid-acrylamide copolymer, acrylamide-acrylic amide-2-methylpropane sulfonate, gums, cellulose derivatives, acrylamide type emulsion thickener, and acrylic acid type emulsion thickener.

6. A cosmetic comprising an ultraviolet light scattering agent coated with an inorganic oxide, and a dispersant.

7. The cosmetic of claim 6, wherein said ultraviolet light scattering agent is selected from the group consisting of ultrafine titanium dioxide, ultrafine zinc oxide, ultrafine iron oxide, ultrafine cerium oxide, and ultrafine cerium phosphate-titanium phosphate complex.

8. The cosmetic of claim 6, wherein said inorganic oxide is selected from the group consisting of silica, alumina and zirconium oxide.

9. The cosmetic of claim 6, wherein said dispersant is a water-soluble polymer.

10. The cosmetic of claim 6, comprising 0.1 to 40 wt % of said ultraviolet light scattering agent coated with an inorganic oxide, calculated as wt % of pure ultraviolet light scattering agent, and 0.01 to 60 wt % of said dispersant, based on the total weight of the cosmetic.

11. The cosmetic of claim 6, further comprising an ultraviolet light absorbing agent.

12. The cosmetic of claim 6, which is a cosmetic of an aqueous solution type, a solubilized type, an emulsion type, a dispersed powder type, a water-oil bilayer type, or a water-oil-powder type.

13. The cosmetic of claim 6, wherein said coated ultraviolet light scattering agent is dispersed homogeneously in the cosmetic without aggregation, thereby showing an improved effect of ultraviolet light protection.

14. A cosmetic comprising ultrafine titanium dioxide coated with an inorganic oxide, and a dispersant, wherein said inorganic oxide is one or more of silica, alumina, and zirconium oxide.

15. The cosmetic of claim 14, wherein said dispersant is alkyl modified carboxyvinylpolymer.

16. The cosmetic of claim 15, further comprising an ultraviolet light absorbing agent.
